(19) United States
(12) Patent Application Publication
(10) Pub. No.: US 2021/0093748 A1
(43) Pub. Date: Apr. 1, 2021

(54) LIQUID BAND-AID CONTAINING PEPTIDE ANTI-INFLAMMATORY ACTIVE INGREDIENT AND PREPARATION METHOD THEREOF

(71) Applicant: ZHEJIANG OCEAN UNIVERSITY, Zhoushan (CN)

(72) Inventors: HONGYU LUO, Zhoushan (CN); XINWEI YU, Zhoushan (CN); ZHIGAO WANG, Zhoushan (CN); JU HUANG, Zhoushan (CN); PENG GAO, Zhoushan (CN); CHONG YI, Zhoushan (CN)

(73) Assignee: ZHEJIANG OCEAN UNIVERSITY

(21) Appl. No.: 17/033,742

(22) Filed: Sep. 26, 2020

(30) Foreign Application Priority Data
Sep. 26, 2019 (CN) 201910918442.7

(51) Int. Cl. A61L 26/00

(52) U.S. Cl. CPC A61L 26/0047; A61L 26/0066; A61L 2300/412; A61L 2300/252; A61L 2300/41; A61L 26/0095

(57) ABSTRACT

The present invention provides a liquid band-aid containing peptide anti-inflammatory active ingredient and a preparation method thereof, which belong to the technical field of medical materials. The liquid band-aid includes film-forming agent, plasticizer, anti-inflammatory substance, and solvent, where the plasticizer includes glycerin, the solvent includes deionized water, and the anti-inflammatory substance includes high F-value oligopeptide with an amino acid sequence of Leu-Leu-Phe-Thr-Thr-Gln and a molecular weight of 736.52 Da. The liquid band-aid can promote the expression of interleukin 10 (IL-10) and inhibit the expressions of cytokine interleukin 6 (IL-6) and tumor necrosis factor (TNF-α), and thus have good anti-inflammatory activity. The liquid band-aid further enhances the close contact between gel and an injured skin surface, increases the cleanliness of a wound surface, and improves the clearance rate of inflammatory cells.

### CROSS-REFERENCE TO RELATED APPLICATIONS

This application claims priority to Chinese Patent Application No. 201910918442.7 with a filing date of Sep. 26, 2019. The content of the aforementioned application, including any intervening amendments thereto, are incorporated herein by reference.

### TECHNICAL FIELD

The invention relates to the technical field of medical materials, and particularly relates to a liquid band-aid containing peptide anti-inflammatory active ingredient and a preparation method thereof.

### BACKGROUND

Traditional Band-Aid has a history of nearly 100 years, and has made a tremendous contribution to the convenience of wound management. The traditional band-aid can cover the wound surface to avoid the influence of the external environment on the wound healing process. The traditional band-aid can realize compressed hemostasis and isolated sterilization, promote wound healing and is easy to carry. Although the traditional band-aid is popular for long, we also feel its disadvantages in special situations in daily life: when treating a particularly complex wound surface, the traditional band-aid cannot be applied well on the wound surface; the secretions and sweat around a human wound cannot be excreted from the body well due to poor breathability of an adhesive tape, and thus will soak the wound, which is disadvantageous for smooth wound healing; some traditional band-aids are asserted to be waterproof, but the waterproof performance thereof is unsatisfactory. The external water will always soak the adhesive tape and a drug-containing layer, and then penetrate into the wound, resulting in wound infection. Liquid band-aid is a translucent protective film by dissolving a film-forming material in a solvent, and adhering the solvent tightly to the wound of the skin by painting or spraying. It has the advantages of bacteria isolation, breathability, waterproofness, ease of use, ease of observing wound conditions and promoting wound recovery. Liquid band-aids include two types, one of which is an over-the-counter skin protectant, which can protect skin abrasions and chronic bedsores and the like; the other is a tissue adhesive used for surgical sutures to treat severe skin tears. Compared to the traditional band-aids, the liquid band-aids have epoch-making significance. However, as far as the current domestic research and market are concerned, the common problem is that it is severely irritating and has a certain odor when used, and the waterproof and breathable performances also need to be further improved.

The prior art, for example, Chinese invention granted patent No. CN 101381356 B, discloses a liquid band-aid. Its raw material composition and ratio include: an active substance, where the active substance include cytokines 0.0042-0.0095%, and the cytokines are at least two selected from EGF, PDGF, FGF and IL-10; a film-forming material, where the film-forming material includes chitosan hydrochloride 2%-8%; and a solvent, where the solvent is deionized water or distilled water. The invention further discloses a method for preparing a liquid band-aid. The liquid band-aid containing multiple biologically-active substances prepared by the invention and/or prepared by the method of the invention can maintain the activity of various biologically-active substances, and can efficiently perform effective and traceless repair for the wounds.

### SUMMARY

The object of the present invention is to provide a liquid band-aid containing peptide anti-inflammatory active ingredient, which can promote the expression of interleukin 10 (IL-10) and inhibit the expressions of proinflammatory cytokine interleukin 6 (IL-6) and tumor necrosis factor (TNF-α), producing good anti-inflammatory activity. Further, the liquid band-aid can enhance the close contact between gel and an injured skin surface, increase the cleanliness of a wound surface, and thus be able to increase a clearance rate of inflammatory cells.

The technical solutions adopted by the present invention to achieve the above objectives are described as follows.

A liquid band-aid containing peptide anti-inflammatory active ingredient is provided, which includes film-forming agent, plasticizer, anti-inflammatory substance, and solvent.

The plasticizer includes glycerin, the solvent includes deionized water, the anti-inflammatory substance includes a high F-value oligopeptide with an amino acid sequence of Leu-Leu-Phe-Thr-Thr-Gln (SEQ ID NO.1), and a molecular weight of 736.52 Da. The high F-value oligopeptide with the amino acid sequence of Leu-Leu-Phe-Thr-Thr-Gln and the molecular weight of 736.52 Da has good anti-inflammatory activity, can promote the expression of interleukin 10 (IL-10), and inhibit the expressions of proinflammatory cytokine interleukin 6 (IL-6) and tumor necrosis factor (TNF-α). The use of the liquid band-aid containing the high F-value oligopeptide can reduce the occurrence of inflammation and promote wound healing.

Preferably, the film-forming agent includes polyvinyl alcohol and modified chitosan. The wound healing process is a complex process involving multiple mechanisms. At present, no single material can meet the complex needs of the wound healing process. The polyvinyl alcohol/modified chitosan bio-composite hydrogel has good absorption and good biocompatibility, biological activity, isolation performance and mechanical strength.

Preferably, the modified chitosan is hydroxycinnamic acid modified chitosan, and dihydroxycoumarin is grafted on the hydroxycinnamic acid modified chitosan. The modified chitosan provided by the invention can improve the water absorption of the liquid band-aid, and can adjust a temperature sensitivity at the same time so as to maintain a gel forming temperature at about 36.5° C., enhance the close contact between the gel and the injured skin surface, and increase the wound cleanliness, increase the clearance rate of inflammatory cells, reduce the inflammatory response of the wound surface, and accelerate wound healing.

Preferably, the specific method for modifying the above chitosan by hydroxycinnamic acid is as follows:

adding dimethyl sulfoxide into chitosan, stirring, then slowly dropping alkaline solution, and alkalinizing for 1.8-2.2 h by stirring; and

dissolving hydroxycinnamic acid in dimethyl sulfoxide, slowly dropping into the solution prepared in the above step while stirring continuously during the dropping, then reacting at 58-62° C. for 5.5-6 h, suction filtering after cooling, washing with deionized water, absolute ethanol and acetone, and drying to obtain hydroxycinnamic acid modified chitosan.

Preferably, the above-mentioned liquid band-aid is prepared by a solution blending method. The polyvinyl alcohol/modified chitosan composite hydrogel prepared by the solution blending method has good antibacterial performance and good coating performance, and has no toxic and side effects on cells.

Another object of the present invention is to provide a method for preparing a liquid band-aid containing peptide anti-inflammatory active ingredient. The preparation steps and conditions of the liquid band-aid are as follows:

based on weight, the above-mentioned liquid band-aid including 50-70 parts of film-forming agent, 1-1.2 parts of high F value oligopeptide, 80-90 parts of plasticizer, and 150-200 parts of solvent;

dissolving the above film-forming agent in the solvent, stirring until completely dissolved, adding high F-value oligopeptide, adding plasticizer, and stirring evenly to prepare the liquid band-aid of the present invention.

The liquid band-aid prepared according to the method provided by the invention has good fluidity, excellent adhesion, small skin irritation, good biocompatibility, good biological activity, good isolation performance and good mechanical strength.

Preferably, the high F-value oligopeptide is a natural oligopeptide.

Preferably, a raw material for preparing the natural oligopeptide is tuna scraps.

Preferably, the preparation method of the natural oligopeptide includes:

using double enzymes to hydrolyze tuna protein step by step: at the first step, hydrolase is pepsin and at the second step, hydrolase is flavor protease;

removing aromatic amino acid; and

isolating and purifying.

Pepsin and flavor protease are used to hydrolyze tuna protein, which is beneficial to improving the enzymatic hydrolysis efficiency and releasing aromatic amino acid. The F value of the resulting protein hydrolysate is high, and a high F-value oligopeptide having an amino acid sequence is Leu-Leu-Phe-Thr-Thr-Gln and a molecular weight of 736.52 Da can be obtained.

Preferably, activated carbon is used to remove the aromatic amino acid.

The beneficial effects of the present invention are described below.

1) In the present invention, the liquid band-aid includes a high F value oligopeptide with an amino acid sequence of Leu-Leu-Phe-Thr-Thr-Gln and a molecular weight of 736.52 Da, which has good anti-inflammatory activity and can promote expression of interleukin 10 (IL-10), inhibit the expressions of cytokine interleukin 6 (IL-6) and tumor necrosis factor (TNF-α), reduce the occurrence of inflammation during wound recovery, and promote wound healing.

2) The present invention can improve the water absorption of the liquid band-aid by modifying chitosan, and can adjust the temperature sensitivity at the same time, so as to maintain the gel-forming temperature at about 36.5° C., enhance the close contact between the gel and the injured skin surface, increase the cleanliness of the wound surface and the clearance rate of inflammatory cells, reduce the inflammatory response of the wound surface, and accelerate wound healing.

### BRIEF DESCRIPTION OF THE DRAWINGS

FIG. 1 is a gel-forming temperature and a water absorption rate of a liquid band-aid according to a test example 1 of the present invention.

FIG. 2 is aggregation of neutrophils in a skin wound according to a test example 2 of the present invention.

FIG. 3 is the number of aggregated neutrophils on a 0.09 mm² wound surface according to a test example 2 of the present invention.

FIG. 4 is an immunoblot of IL-10, IL-6 and TNF-α according to a test example 2 of the present invention.

FIG. 5 is relative expression levels of IL-10, IL-6 and TNF-α according to a test example 2 of the present invention.

FIG. 6 is a healing rate of a wound surface by treating with a liquid band-aid according to a test example 2 of the present invention.

### DETAILED DESCRIPTION OF THE EMBODIMENTS

The present invention will be further described in detail in conjunction with the following examples.

### Example 1

A method for preparing a liquid band-aid containing peptide anti-inflammatory active ingredient includes:

using double enzymes to hydrolyze tuna protein step by step: taking tuna minced meat, where the enzyme for hydrolysis of the first step was pepsin, the enzyme amount added was 600 U/g with pH being 2.0, the ratio of feed and water being 1:7, the temperature being 35° C., and the hydrolysis time being 3 h; In the second step, the enzyme for hydrolysis being flavor protease, and the enzyme amount added being 50,000 U/g, with pH 6.5, the temperature 50° C., and the hydrolysis time 4 h, and thus obtaining protein hydrolysate;

removal of aromatic amino acid: filtering the protein hydrolysate under vacuum, adding 200 mesh activated carbon powder at a ratio of solid to liquid of 1:20 with pH 3.0, temperature 35° C. and time 3 h; adsorbing the aromatic amino acid in a static state; after adsorption, centrifuging at 4000 rpm for 10 min, and taking supernatant to obtain an oligopeptide solution;

gel filtration: concentrating the oligopeptide solution after static dearomatization with activated carbon, lyophilizing, then taking 50 mg for dissolution in 1.5 ml distilled water, and separating and purifying with Sephadex G-25 dextran gel chromatography column (1.6×50 cm); after loading sample, eluting with pH7.2 phosphate buffer, collecting one tube of eluent every 230 seconds with each tube being 3 ml, and measuring the ultraviolet absorbance (A) of each tube at 220 nm and 280 nm to obtain four components A1, A2, A3, A4, respectively detecting the amino acid composition and content of each component, and calculating the F value of each component according to the following formula:

F=(Val+Ile+Leu)/(Tyr+Phe)

in the above formula, Val, Ile, Tyr, Phe, Leu respectively represents the amounts of valine, isoleucine, tyrosine, phenylalanine and leucine in the unit of mg/mL;

Calculations show that the F value of A3 was the highest, i.e. 37.52.

Purification of oligopeptide by reverse high-performance liquid chromatography: concentrating the A3 component oligopeptide solution after gel separation and lyophilizing; taking 1 mg to be dissolved to 1 ml with ultrapure water containing 0.05% TFA, centrifuging, taking supernatant, and loading RP-HPLC chromatography; chromatographic conditions: injection volume 500 μL, flow rate 0.8 ml/min, detection wavelength 280 nm, column temperature 25° C., mobile phase being phase A-B, where the phase A was ultrapure water containing 0.05% trifluoroacetic acid, and the phase B was acetonitrile containing 0.05% trifluoroacetic acid; gradient elution conditions (phase B): 0-9 min, 0% B; 9-40 min, 0%-100% B; 40-50 min, 100% B; finally, collecting four components M1, M2, M3 and M4 on a chromatographic peak, lyophilizing, weighing, determining the amino acid sequence of the collected components, and accurately determining the molecular weight of each component, obtaining the M3 component as a target oligopeptide with an amino acid sequence Leu-Leu-Phe-Thr-Thr-Gln and a molecular weight of 736.52 Da;

preparation of modified chitosan: adding 1.5 g of chitosan into a three-necked flask, adding 10 mL of dimethyl sulfoxide, stirring and swelling at 30° C. for 1 h, slowly dropwise adding alkaline solution, and alkalinizing for 2 h by stirring; taking 3 g of hydroxycinnamic acid to be dissolved in dimethyl sulfoxide, slowly dropping into the flask, while stirring continuously during the dropping addition, then reacting at 60° C. for 5.8 h, after that, cooling, suction filtrating, fully washing with deionized water, absolute ethanol and acetone in sequence, and drying to obtain hydroxycinnamic acid modified chitosan;

dissolving 1 g of hydroxycinnamic acid-modified chitosan in 100 mL of 2% acetic acid solution, swelling for 2 h, adding into the alkaline solution for precipitation while stirring, suction filtering, washing with acetone, suction-filtering to half-dry, transferring to 100 mL of acetone, stirring into a suspension, dropping 5 mL of epichlorohydrin therein, and adjusting the temperature to 35° C. and reacting for 24 h; then adding 3 mL of dihydroxycoumarin, reacting at 60° C. for 6 h, and then adding 8 mL of dihydroxycoumarin, 50 mL NaOH solution, 0.05 g potassium iodide, stirring for 4 h, cooling and suction filtering, and then washing by deionized water, absolute ethanol and acetone thoroughly and drying to obtain dihydroxycoumarin grafted modified chitosan;

preparation of the liquid band-aid: dissolving 16 g of polyvinyl alcohol in 160 g of deionized water, stirring at 90° C. in water bath until completely dissolved, adding 40 g of modified chitosan, after complete dissolution, adding 1 g of high F value oligopeptide and 64 g of glycerin; after uniformly mixing, preparing the liquid band-aid of the present invention.

### Comparative Example 1

The high F value oligopeptide was not added to the liquid band-aid, and the rest was completely the same as in Example 1.

### Comparative Example 2

Chitosan was not modified with hydroxycinnamic acid, and the rest was exactly the same as in Example 1.

### Comparative Example 3

Chitosan was not grafted with dihydroxycoumarin, and the rest was exactly the same as in Example 1.

### Comparative Example 4

Chitosan was not modified with hydroxycinnamic acid, nor grafted with dihydroxycoumarin, and the rest was completely the same as in Example 1.

### Comparative Example 5

Chitosan was not modified with hydroxycinnamic acid, nor grafted with dihydroxycoumarin, no high F value oligopeptide was added to the liquid band-aid, and the rest was completely the same as in Example 1.

### Test Example 1

Detection of Temperature Sensitivity of Liquid Band-Aid:

The prepared liquid band-aid was placed in a water bath environment and gradually heated at a rate of 0.5° C./min. After each temperature increase, the solution system by inversion was observed. If the system was inverted with no liquid flowing out, the temperature was the lowest gel forming temperature.

Detection of Water Absorption Rate of Liquid Band-Aid:

The prepared liquid band-aid was gelled in a water bath, and the film was placed in a PBS solution and swelled and balanced at room temperature. After The wet film surface was dried and weighed. After drying and weighing the wet film, the formula for calculating the water absorption rate of the gel was as follows:

Water absorption rate=(W−W0)/W0×100%

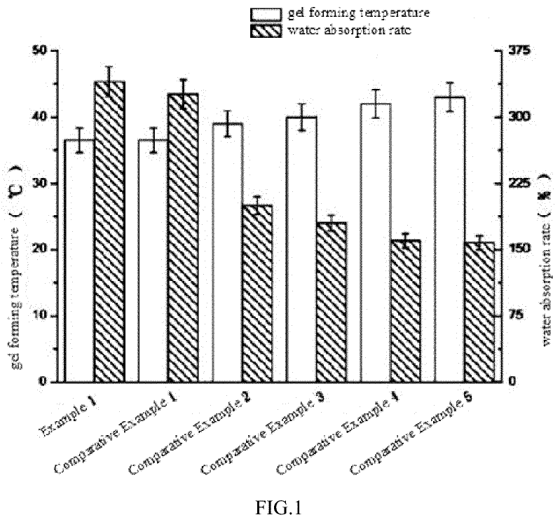

In the above formula, W is a mass of wet gel at the time of swelling balancing; W0 is a mass of dry gel. FIG. 1 shows a gel forming temperature and a water absorption rate of liquid band-aid.

It can be seen from FIG. 1 that the gel forming temperatures of Example 1 and Comparative Example 1 are about 36.5° C., which is within a body temperature range of the human body. The gel forming temperatures of Comparative Example 2, Comparative Example 3, Comparative Example 4, and Comparative Example 5 are higher than the human body temperature, and the water absorption rates of Example 1 and Comparative Example 1 are significantly higher, which show that chitosan modified with hydroxycinnamic acid, or grafted by dihydroxycoumarin can improve the water absorption of the liquid band-aid, so as to maintain the gel forming temperature at about 36.5° C.

### Test Example 2

Building of a mouse skin resection wound model: intraperitoneally injecting 100 μL of 2% pentobarbital sodium, removing mouse hair, culturing in a dry and clean environment for 24 h, and making a total skin resection wound with a diameter of 8 mm on both sides of the back of the mouse respectively by using a puncher, where the wounds for which no treatment is performed were taken as a control group, and the untreated wounds were taken as a blank group.

The treated mice were cultured in a dry and clean environment, photographs were taken after the second day of culture, and wound tissues (including normal tissues about 5 mm away from the wound surface) were extracted for subsequent experiments.

Wound Surface Neutrophil Detection:

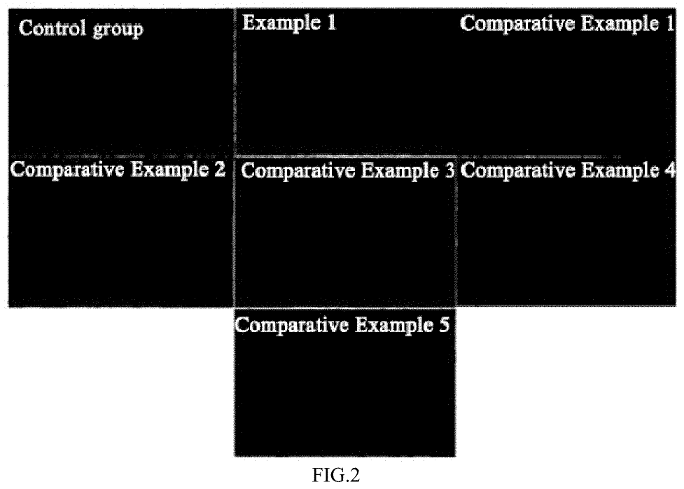

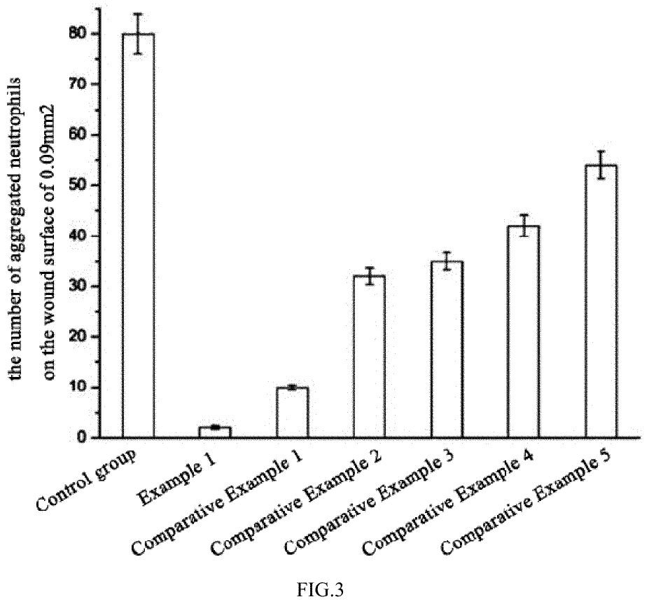

The skin tissue was embedded and sliced, and a rabbit neutrophil elastase (NE) immunohistochemical detection kit was used to detect the aggregation of neutrophils. The aggregation of neutrophils in a skin wound surface was shown at a scale of 25 μm in FIG. 2. The aggregation number of neutrophils on a wound surface of 0.09 mm² was shown in FIG. 3.

It can be seen from FIG. 3 that the aggregation numbers of neutrophils in the wound surface of 0.09 mm² in Example 1 and Comparative Example 1 are significantly higher than those in Comparative Example 2, Comparative Example 3, Comparative Example 4, and Comparative Example 5, which shows that hydroxycinnamic acid modified chitosan, dihydroxycoumarin grafted chitosan are able to enhance the close contact between the gel and the injured skin surface, increase the cleanliness of the wound surface, increase the clearance rate of inflammatory cells, and reduce the inflammation of the wound surface. The aggregation numbers of neutrophils on the wound surface of 0.09 mm² in the Comparative Example 2, Comparative Example 3 and Comparative Example 4 were significantly higher than that of Comparative Example 5, which shows that the liquid band-aid containing the high F-value oligopeptide with an amino acid sequence Leu-Leu-Phe-Thr-Thr-Gln and a molecular weight of 736.52 Da can reduce the occurrence of inflammation, which was attested by the aggregation of neutrophils in the skin wound as shown in FIG. 2.

The expressions of interleukin 10 (IL-10), interleukin 6 (IL-6) and tumor necrosis factor (TNF-α) were detected by immunoblotting:

using β-actin as a reference protein, and detecting the expressions of IL-10, IL-6 and TNF-α by WB immunoblotting kit.

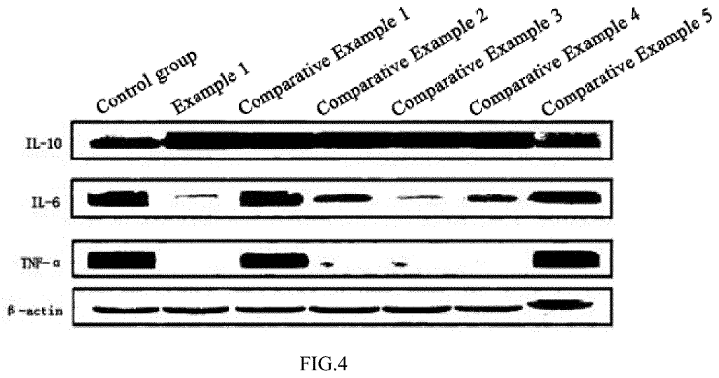

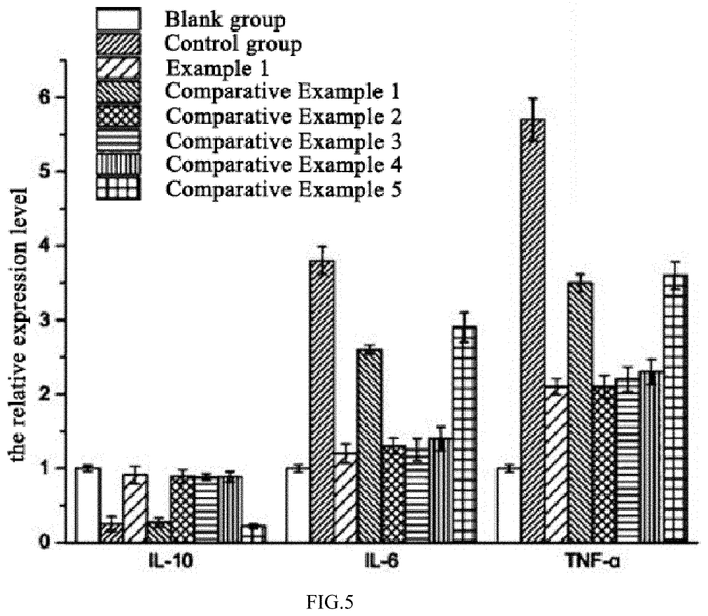

FIG. 4 shows the immunoblots of IL-10, IL-6 and TNF-α, and FIG. 5 shows the relative expression levels of IL-10, IL-6 and TNF-α.

It can be seen from FIG. 4 and FIG. 5 that the relative expression levels of IL-10 in Example 1, Comparative Example 2, Comparative Example 3, and Comparative Example 4 are significantly higher than those in Comparative Example 1 and Comparative Example 5, and the relative expressions level of IL-6 and TNF-α are significantly lower than those of Comparative Example 1 and Comparative Example 5. It is indicated that the liquid band-aid containing the high F value oligopeptide with the amino acid sequence Leu-Leu-Phe-Thr-Thr-Gln and the molecular weight of 736.52 Da is able to promote the expression of interleukin 10 (IL-10), and inhibit the expressions of the proinflammatory cytokine interleukin 6 (IL-6) and tumor necrosis factor (TNF-α) so as to reduce the occurrence of inflammation.

Detection of Wound Surface Healing Process:

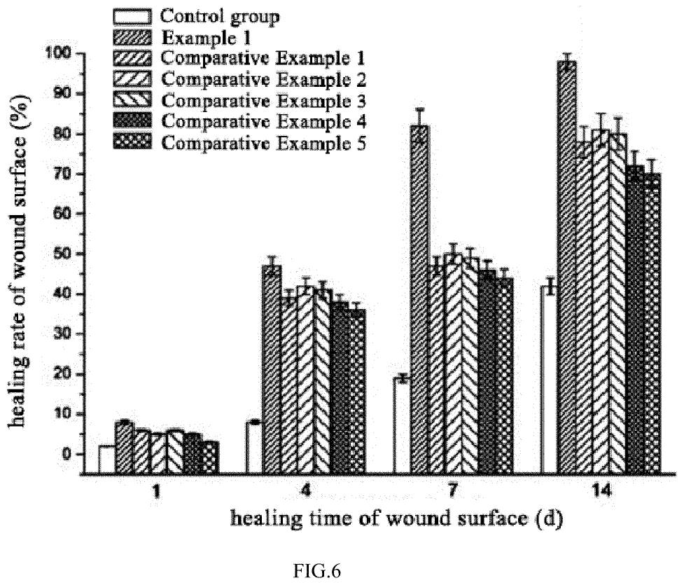

On the 1st, 4th, 7th, and 14th days after treatment, the wound surface healing after treating the total resection wound with the liquid band-aid was observed. The wound healing rate is shown in FIG. 6.

It can be seen from FIG. 6 that the healing rate of Example 1 is significantly higher than those of Comparative Example 1, Comparative Example 2, and Comparative Example 3, and the healing rate of Comparative Example 4 is significantly higher than that of Comparative Example 5, which shows that the liquid band-aid containing the high F value oligopeptide can reduce the occurrence of inflammation and promote the healing of wound surface. The healing rate of Example 1 is significantly higher than those of Comparative Example 2, Comparative Example 3 and Comparative Example 4, and the healing rate of Comparative Example 1 is significantly higher than that of Comparative Example 5, which shows that chitosan modified with hydroxycinnamic acid or grafted with dihydroxycoumarin can reduce the inflammatory response of the wound surface, improve the curative effect and accelerate wound healing.

The conventional approach in the above examples is the prior art known to those skilled in the art, and thus will not be described in detail here.

The above examples are only used to illustrate the present invention, rather than limit the present invention. Those of ordinary skill in the art can make various changes and modifications without departing from the spirit and scope of the present invention. Therefore, all equivalent technical solutions also belong to the scope of the present invention, and the protection scope of the present invention should be defined by the claims.

1. A liquid band-aid containing peptide anti-inflammatory active ingredient, comprising film-forming agent, plasticizer, anti-inflammatory substance and solvent, wherein the plasticizer comprises glycerin, the solvent comprises deionized water, the anti-inflammatory substance comprises high F-value oligopeptide with an amino acid sequence of Leu-Leu-Phe-Thr-Thr-Gln (SEQ ID NO.1) and a molecular weight of 736.52 Da.

2. The liquid band-aid according to claim 1, wherein the film-forming agent comprises polyvinyl alcohol and modified chitosan.

3. The liquid band-aid according to claim 2, wherein the modified chitosan is hydroxycinnamic acid modified chitosan, and dihydroxycoumarin is grafted on the hydroxycinnamic acid modified chitosan.

4. The liquid band-aid according to claim 3, wherein, a specific method for modifying chitosan by hydroxycinnamic acid comprises:
1) adding dimethyl sulfoxide into chitosan, stirring, then slowly dropping alkaline solution, and alkalinizing for 1.8-2.2 h by stirring;
2) dissolving hydroxycinnamic acid in dimethyl sulfoxide, slowly dropping into the solution prepared in step 1) while stirring continuously during the dropping, then reacting at 58-62° C. for 5.5-6 h, suction-filtering after cooling, fully washing with deionized water, absolute ethanol and acetone in sequence, and drying to obtain hydroxycinnamic acid modified chitosan.

5. The liquid band-aid according to claim 1, wherein the liquid band-aid is prepared by a solution blending method.

6. A method for preparing the liquid band-aid according to claim 1, wherein the steps and conditions for preparing the liquid band-aid are as follows: based on weight, the liquid band-aid comprising 50-70 parts of film-forming agent, 1-1.2 parts of high F value oligopeptide, 80-90 parts of plasticizer, and 150-200 parts of solvent; dissolving the film-forming agent in the solvent, stirring until completely dissolved, adding high F-value oligopeptide, adding plasticizer, and stirring evenly to prepare the liquid band-aid of the present invention.

7. The method according to claim 6, wherein, the high F-value oligopeptide is a natural oligopeptide.

8. The method according to claim 7, wherein, a raw material for preparing the natural oligopeptide is tuna scraps.

9. The method according to claim 8, wherein, the method of preparing the natural oligopeptide comprises:
1) using double enzymes to hydrolyze tuna protein step by step: at the first step, hydrolase is pepsin, and at the second step, hydrolase is flavor protease; 2) removing aromatic amino acid; 3) isolating and purifying.

10. The method according to claim 9, wherein the aromatic amino acid is removed by using activated carbon.


SEQUENCE LISTING (1 sequence)

SEQ ID NO: 1 - protein, 6 aa, Artificial Sequence
  Synthesized
LLFTTQ